(19) United States
(12) Patent Application Publication
(10) Pub. No.: US 2003/0096012 A1
(43) Pub. Date: May 22, 2003

(54) Film-forming powder, compositions containing it, methods for their preparation and their uses

(76) Inventors: Jerome Besse, Listrac Medoc (FR); Laurence Besse, Listrac Medoc (FR); Philippe Cornu, Ableiges (FR); Brigitte Taravella, Paris (FR)

Correspondence Address: Michael L. Kenaga, Piper Marbury Rudnick & Wolfe, P.O. Box 64807, Chicago, IL, 60664-0807, US

(21) Appl. No.: 10/097,781

(22) Filed: Mar. 14, 2002

(30) Foreign Application Priority Data
Nov. 21, 2001 (FR) 0115069

(51) Int. Cl. A61K 38/28; A61K 38/21; A61K 31/7048; A61K 31/56; A61K 31/5513

(52) U.S. Cl. 424/489; 514/29; 514/170; 514/171; 514/254.07; 514/570; 514/383; 514/269; 514/2; 514/411; 514/3; 514/561; 514/573; 514/221; 514/252.17; 514/649; 424/85.4

(57) ABSTRACT

The present invention relates to a film-forming powder comprising at least one active substance, at least one bioadhesive agent, and optionally surfactants, wetting agents, plasticizers, binders, retardants, penetration promoters and bioerodible diluents.

The present invention relates to a film-forming powder, pharmaceutical, cosmetic or nutraceutical compositions containing this powder, as well as to methods for their manufacture and their uses.

The film-forming powder according to the present invention possesses the specific feature of forming a film in situ at the time of its application. It can be applied to the dermis and/or to a mucous membrane.

Due to the fact that this powder forms a film on the dermis or on the mucous membrane during its application, it allows the sustained prolonged release of the active substance(s) which it contains. This sustained release can occur in several ways. For example, linearly or with a “burst effect” (immediate release of part of the active substance), also called “bimodal release profile” or “rapid and sustained release effect”.

A decisive advantage of this galenic form consists in the fact that the film erodes with time so as to leave no residue.

Fluid compositions capable of forming films in situ during their application are already known. Thus, U.S. Pat. No. 5,081,157 and U.S. Pat. No. 5,081,158, and patent applications WO 96/30000, WO 97/31621, WO 00/10540, WO 00/38658, WO 01/13955 and WO 01/43722 describe film-forming compositions for transdermal and/or transmucosal application. These compositions may be in the form of a solution, a suspension or a gel.

The film-forming compositions already known in the prior art suffer from numerous disadvantages. Among these, there may be mentioned the difficulties of preparation linked to the production of compositions which can then form a homogeneous film, the difficulties of storing these galenic forms because they are often unstable, and the difficulties linked to their administration.

In particular, liquids, just like gels, are difficult to position precisely on the dermis or the mucous membranes, and tend to slide or to move.

The applicant companies have therefore sought to develop a galenic form which can overcome the disadvantages encountered by the earlier formulations.

They have thus succeeded in developing a film-forming powder which allows the in situ formation of a film having good adhesive and cohesion properties.

The film-forming composition in the form of a powder according to the invention, unlike the fluid products of the prior art, does not require the use of any solvents during the administration of the product. This is quite obviously a decisive advantage for a product for pharmaceutical, cosmetic or nutraceutical use. The powder form also allows a very good stability of the product during storage, greater than that of products in the form of solutions, suspensions or gels.

The film-forming powder according to the present invention therefore has numerous advantages compared with galenic forms known in the prior art.

Accordingly, the present invention relates to a film-forming powder comprising at least one active substance, at least one bioadhesive agent, and optionally surfactants, wetting agents, plasticizers, binders, hydrophilic or nonhydrophilic retardants, penetration entrancers and bioerodible diluents.

The active substances of the film-forming powder according to the invention may be selected from those conventionally used in the following specialities: allergology, anaesthetic/intensive care, cancerology and haematology, cardiology and angiology, contraception and abortion, dermatology, endocrinology, gastroenterohepatology, gynaecology, immunology, infectiology, metabolism and nutrition, neurology/psychiatry, ophthalmology, ear, nose and throat, pneumology, rheumatology, stomatology, toxicology, urology/nephrology, and from analgesics and antispasmodics, anti-inflammatory agents, contrast products used in radiology, haemostatics, and products for treating blood and derivatives.

Advantageously, the active substances may be selected from the group consisting of the active substances crossing the skin barrier and reaching the systemic circulation, such as cyproterone acetate, Δ-⁴-androstenedione, 3-ketodesogestrel, desogestrel, gestodene, estradiol and its derivatives, norethisterone acetate, progesterone, testosterone, trinitrine, fentanyl, nitroglycerine, nicotine (S(-)-nicotine), scopolamine, clonidine, isosorbide dinitrate, levonorgestrel in combination with ethinylestradiol or with estradiol, androstanolone, alclometasone dipropionate, and combinations thereof.

They may also be selected from the active substances crossing the skin barrier and having a localized action such as: acetazolamide, acyclovir, adapalene, alclomethasone dipropionate, amcinonide, ameleine, bamethan sulphate+escin, betamethasone valerate, betamethasone dipropionate, bufexamac, caffeine, calcipotriol monohydrate, cetrimonium bromide, clobetasol propionate, crilanomer, desonide, dexpanthenol, diclofenac, diflucortolone, valerate, difluprednate, diphenydramine hydrochloride, econazole nitrate, erythromycin, flumetasone pivalate, fluocinolone acetonide, fluocinodine, fluocortolone, fluocortolone hexanoate, fluocortolone pivalate, hydrocortisone, hydrocortisone acetate, ibacitabin, ibuprofen, imiquimod, ketoconazole, ketoprofen, lidocaine, metronidazole, miconazole nitrate, minoxidil, niflumide acid, penciclovir, benzoyl peroxide, piroxam, iodinated povidone, promestriene, pyrazonibutasone, roxithromycin, sulphacetamide, triamconolone, tazarotene, tretinoin and isotretinoin, triclocarban, vidarabine monophosphate and combinations thereof.

They may also be selected from the following active substances: □β₃-adrenergic agonist, growth hormone, oxybutinin, buprenorphine, pergolide, estradiol+nestorone, nestorone, 7α-methyl-19-nortesterone, mecamylamine (antagonist of nicotine)+nicotine, salbutamol, selegiline, buspirone, ketotifen, lidocaine, testosterone+estradiol, ketorolac, eptazocine, insulin, α-interferon, prostaglandines, 17β-estradiol+norethindrone acetate, 5-aminolevulinic acid, benzodiazepine alprozolam, diclofenac, fenoprofen, flubiprofen, ketoprofen, methyl phenidate, miconazole, piroxicam, bruprenorphine, alprozolam, dexmedetomidine, prazosin (α-adrenergic antagonist), gestodene+ethinylestradiol, alprostadil, tulobuterol (β-adrenergic agonist), ethinylestradiol+norelgestromin, ketorolac, physostigmine, lidocaine, medindolol (α-adrenergic agonist), rotigotine (D2 dopamine antagonist), ethinylestradiol+norethindrone acetate, thiatolserine, and combinations thereof.

They may also be selected from the following active substances: esomeprazole, melagatran (in case of thrombosis), rosuvastatin, ezetimide, pitavastatin (hyperlipidaemia), mitiglinide (type II diabetes), cilomilast, viozan (asthma), aripipazole (psychiatry), omapatrilat (hypertensive), orzel (cancerology), caspofungin acetate, voriconazole (infections), novel COX inhibitors such as etoricoxib (inflammation), valdecoxib (arthritis) and parecoxib, substance P antagonist (depression), darifenacin (urology), eletriptan (migraine), alosetron, tegaserod, capravirine (HIV) and combinations thereof.

The film-forming powder may contain one or more active substances, combined with each other.

For cosmetic applications, the active substance may be chosen from the group comprising emollients, moisturizing agents, vitamins, complexes of fruit amino acids and the like.

For nutraceutical applications, the active substance may be chosen from the group comprising vitamins, inorganic salts, beer yeast and the like.

According to a preferred embodiment of the powder according to the invention, the active substances are micronized before being mixed with the other ingredients. It is also possible to mix the nonmicronized active substance with the other ingredients of the powder and then to micronize the final mixture. This promotes the homogeneity of the film and the cohesion and adhesion of the particles. Moreover, systems for spraying powder are particularly well suited to the spraying of micronized products.

The bioadhesive agent of the film-forming powder according to the invention is advantageously selected from the group consisting of methyl cellulose, carboxymethyl cellulose, carboxymethyl cellulose sodium, hydroxyethyl cellulose, hydroxypropyl cellulose, hydroxypropyl methyl cellulose sodium, polyvinylpyrrolidone, polyvinyl alcohols, polyisobutylene, polyisopropene, xanthan gum, locust bean gum, chitosan, polycarboxylates, carbomers such as carbopol, acrylic/methacrylic acid copolymer, acrylic acid/acrylamide copolymer, acrylic acid/methyl methacrylate copolymer, acrylic acid/polyethylene glycol copolymer, polyacrylic acid/butyl acrylate copolymer, HEMA (2-hydroxyethyl methacrylate) copolymerized with Polymeg® (polytetramethylene glycol), Cydot® marketed by 3M (carbopol combined with polyisobutylene), pectin (of low viscosity), polyethylene oxide, methyl vinyl ether/maleic anhydride copolymer, tragacanth, monomethyl ether, monomethacrylate, drum dried waxy maize starch, sodium stearyl fumarate, sodium hyaluronate, guar gum, sodium alginate, starches, dextran and mixtures thereof.

The powder according to the invention may also comprise one or more surfactants, which are preferably nonionic, such as polyoxyethylene sorbitan (fatty acid ester, polyoxyethylene alkyl ether, polyoxyethylene derived from castor oil and mixtures thereof.

If necessary, this powder may also comprise a wetting agent selected from the group consisting of polyols such as sorbitol, or glycerine, such as PEG and mixtures thereof.

The powder according to the invention may also comprise a plasticizer selected from the group consisting of dibutyl phthalate, dibutyl sebacate, acetyltributyl citrate, acetyltriethyl citrate, tributyl citrate, tributylethyl citrate, triacetin, PEG, propylene glycol, glycerol, glycerol monoesters and derivatives, castor oil and mixtures thereof.

The powder according to the invention may also comprise a binder selected from the group consisting of acacia, alginic acid, carboxymethyl cellulose sodium, microcrystalline cellulose, dextrins, ethyl cellulose, gelatin, glucose, guar gum, hydroxypropyl methyl cellulose, methyl cellulose, polyethylene oxide, povidone, pregelatinized starch and mixtures thereof.

The powder according to the invention may also comprise a hydrophilic or nonhydrophilic retardant selected from the group consisting of hydroxypropyl methyl cellulose acetate or succinate, hydroxypropyl methyl cellulose, hydroxypropyl cellulose, ethyl cellulose, hydroxyethyl cellulose, carboxymethyl cellulose sodium, polyvinyl alcohols, hydrocolloids such as: pectins, alginates, guar gum, xanthan gum, gum Arabic, agar, dextrin, carragheenan, polyethylene oxide, carbomers, polymers and copolymers of acrylic acid, of methyl methacrylate, of polyvinyl acetate, of carboxymethyl acetate, hydrogenated castor oil and derivatives, bentonite and derivatives, and mixtures thereof.

The powder according to the invention may also comprise a bioerodible diluent selected from the group consisting of calcium or sodium carbonate or bicarbonate, sucrose, mannitol, xylitol, sorbitol, lactose, cellulose or microcrystalline cellulose powder, starch and its derivatives, dibasic calcium phosphate, tribasic calcium phosphate, calcium sulphate, dextrates, dextrins, dextrose excipients, fructose, kaolin, lactitol and mixtures thereof.

The film-forming powder according to the invention may also comprise a penetration entrancers which may be selected from the group consisting of aliphatic fatty acid esters such as isopropyl myristate, fatty acids such as oleic acid; alcohols or polyols such as ethanol, propylene glycol and polyethylene glycol; components of essential oils and terpenic derivatives (such as eugenol, geraniol, nerol, eucalyptol, menthol); surfactants; moisturizers such as glycerin, urea; keratolytics such as alpha-hydroxy acids.

According to a preferred embodiment of the film-forming powder according to the invention, it has a particle size of between 0.01 μm and 1000 μm, preferably between 0.1 μm and 100 μm and more preferably still between 1 μm and 50 μm.

The invention also relates to a pharmaceutical, cosmetic or nutraceutical composition comprising the film-forming powder. This composition may be applied to the dermis or the mucous membranes.

When it is administered by the mucosal route, it may be applied, for example, through the buccal mucosa, the nasal mucosa or the vaginal mucosa.

When the powder according to the invention is administered by the transdermal or transmucosal route, it will have a systemic effect or a local effect according to the nature of the active substance and the other components present in the powder.

Advantageously, the composition according to the invention, comprising the film-forming powder, exists in a pulverizable dry form. This allows easy delivery of a precise dose.

The invention also relates to a process for the preparation of a film-forming powder.

All the processes known to persons skilled in the art may be used in the context of the production of this film-forming powder.

There may be mentioned, as an example of a method for preparing a powder: wet or dry granulation, optionally followed by micronization.

Or according to another embodiment, the active substance is micronized and then mixed with the excipients in powdered form, and the mixture thus obtained is granulated, by wet or dry granulation.

According to an advantageous embodiment of the process according to the invention, the active substance alone or the final mixture of ingredients may be micronized.

It is also possible to prepare the powder according to the invention by spray-drying. According to this process, the raw materials are solubilized in a solvent in order to obtain a solution or a suspension which is then spray-dried, for example in a NIRO® type spray-dryer or equivalent. The grain thus obtained may be used directly or after micronization.

The invention will be understood more clearly with the aid of the nonlimiting examples described below.

### EXAMPLE 1

### Film-Forming Powders According to the Invention

Four powders, each having the following composition by weight, are prepared:

| Components | Quantity in % |
|---|---|
| Buprenorphine | 3 |
| CARBOPOL ® 974 PNF | 45 |
| (hypromellose) - Metolose ® | |
| 90SH100 000 SR | 45 |
| Stearic acid | 5 |
| Propylene glycol | 2 |

| Components | Quantity in % |
|---|---|
| 17β-Estradiol | 5 |
| CARBOPOL® 974 PNF | 33 |
| KLUCEL ® - HXF | |
| (Hydroxypropyl cellulose) | 49 |
| Stearic acid | 10 |
| Oleic acid | 3 |

| Components | Quantity in % |
|---|---|
| Molsidomine | 7 |
| CARBOPOL ® 974 PNF | 20.9 |
| Povidone ® K30 | 4.2 |
| HPC | 20.9 |
| Lactose | 20.9 |
| CMC sodium | 20.9 |
| Talc | 4.2 |
| Menthol | 1.0 |

| Components | Quantity in % |
|---|---|
| Salbutamol | 5 |
| HPMC | 45 |
| Carbopol ® 974 PNF | 45 |
| Hydrogenated vegetable oil | 4 |
| Sodium lauryl sulphate | 1 |

The various components are mixed in a mixer-granulator of the vacuum mixer-granulator-drier type ROTOLAB ZANCHETTA or equivalent until the mixture is homogenized. Next, a wetting solution or suspension is incorporated, with stirring, in order to obtain a wet granulate.

This granulate is then dried under suitable conditions so as to evaporate the granulation solvent. This granulate is then calibrated and then micronized according to the most advantageous mode of preparation before being mixed with optional adjuvants and then packaged in a container suited to its mode of application.

1. Film-forming powder comprising at least one active substance, at least one bioadhesive agent, and optionally surfactants, wetting agents, plasticizers, binders, retardants, penetration antrancers and bioerodible diluents.

2. Film-forming powder according to claim 1, wherein the active substance is micronized.

3. Film-forming powder according to either of claim 1, wherein the powder is micronized.

4. Film-forming powder according to claim 1, wherein the active substance is selected from the group consisting of estradiol and its derivatives, norethisterone acetate, progesterone, testosterone, trinitrine, fentanyl, nitroglycerine, nicotine (S (-)-nicotine), scopolamine, clonidine, isosorbide dinitrate, levonorgestrel in combination with ethinylestradiol or with estradiol, androstanolone, alclometasone dipropionate, acetazolamide, acyclovir, adapalene, alclomethasone dipropionate, amcinonide, ameleine, bamethan sulphate+escin, betamethasone valerate, betamethasone dipropionate, bufexamac, caffeine, calcipotriol monohydrate, cetrimonium bromide, clobetasol propionate, crilanomer, desonide, dexpanthenol, diclofenac, diflucortolone, valerate, difluprednate, diphenydramine hydrochloride, econazole nitrate, erythromycin, flumetasone pivalate, fluocinolone acetonide, fluocinodine, fluocortolone, fluocortolone hexanoate, fluocortolone pivalate, hydrocortisone, hydrocortisone acetate, ibacitabin, ibuprofen, imiquimod, ketoconazole, ketoprofen, lidocaine, metronidazole, miconazole nitrate, minoxidil, niflumide acid, penciclovir, benzoyl peroxide, piroxam, iodinated povidone, promestriene, pyrazonibutasone, roxithromycin, sulphacetamide, triamconolone, tazarotene, tretinoin and isotretinoin, triclocarban, vidarabine monophosphate, β₃-adrenergic agonist, growth hormone, oxybutinin, buprenorphine, pergolide, estradiol+nestorone, nesterone, 7α-methyl-19-nortesterone, mecamylamine (antagonist of nicotine)+nicotine, salbutamol, selegiline, buspirone, ketotifen, lidocaine, testosterone+estradiol, ketorolac, eptazocine, insulin, α-interferon, prostaglandines, 17β-estradiol+norethindrone acetate, 5-aminolevulinic acid, benzodiazepine alprozolam, diclofenac, fenoprofen, flubiprofen, ketoprofen, methyl phenidate, miconazole, piroxicam, bruprenorphine, alprozolam, dexmedetomidine, prazosin (α-adrenergic antagonist), gestodene+ethinylestradiol, alprostadil, tulobuterol (β-adrenergic agonist), ethinylestradiol+norelgestromin, ketorolac, physostigmine, lidocaine, medindolol (α-adrenergic agonist), rotigotine (D2 dopamine antagonist), ethinylestradiol+norethindrone acetate, thiatolserine, esomeprazole, melagatran (in case of thrombosis), rosuvastatin, ezetimide, pitavastatin (hyperlipidaemia), mitiglinide (type II diabetes), cilomilast, viozan (asthma), aripipazole (psychiatry), omapatrilat (hypertensive), orzel (cancerology), caspofungin acetate, voriconazole (infections), novel COX inhibitors such as etoricoxib (inflammation), valdecoxib (arthritis) and parecoxib, substance P antagonist (depression), darifenacin (urology), eletriptan (migraine), alosetron, tegaserod, capravirine (HIV) and combinations thereof.

5. Film-forming powder according to any one of claims 1 to 4, characterized in that the bioadhesive agent is selected from the group consisting of methyl cellulose, carboxymethyl cellulose, carboxymethyl cellulose sodium, hydroxyethyl cellulose, hydroxypropyl cellulose, hydroxypropyl methyl cellulose sodium, polyvinylpyrrolidone, polyvinyl alcohols, polyisobutylene, polyisopropene, xanthan gum, locust bean gum, chitosan, chitosan chloride, polycarboxylates, carbomers such as carbopol, acrylic/methacrylic acid copolymer, acrylic acid/acrylamide copolymer, acrylic acid/methyl methacrylate copolymer, acrylic acid/polyethylene glycol copolymer, polyacrylic acid/butyl acrylate copolymer, HEMA (2-hydroxyethyl methacrylate) copolymerized with Polymeg® (polytetramethylene glycol), Cydot® marketed by 3M (carbopol combined with polyisobutylene), pectin (of low viscosity), polyethylene oxide, methyl vinyl ether/maleic anhydride copolymer, tragacanth, monomethyl ether, monomethacrylate, drum dried waxy maize starch, sodium stearyl fumarate, sodium hyaluronate, guar gum, sodium alginate, starches, dextran and mixtures thereof.

6. Film-forming powder according to claim 1, wherein the surfactant is preferably selected from nonionic surfactants such as polyoxyethylene sorbitan (fatty acid ester), polyoxyethylene alkyl ether, polyoxyethylene derived from castor oil, and mixtures thereof.

7. Film-forming powder according to claim 1, wherein the wetting agent is selected from the group consisting of polyols such as sorbitol, glycerin, polyethylene glycol and mixtures thereof.

8. Film-forming powder according to wherein claim 1, wherein the plasticizer is selected from the group consisting of dibutyl phthalate, dibutyl sebacate, acetyltributyl citrate, acetyltriethyl citrate, tributyl citrate, tributylethyl citrate, triacetin, PEG, propylene glycol, glycerol, glycerol monoesters and derivatives, castor oil and mixtures thereof.

9. Film-forming powder according to claim 1, wherein the binder is selected from the group consisting of acacia, alginic acid, carboxymethyl cellulose sodium, microcrystalline cellulose, dextrins, ethyl cellulose, gelatin, glucose, guar gum, hydroxypropyl methyl cellulose, methyl cellulose, polyethylene oxide, povidone, pregelatinized starch and mixtures thereof.

10. Film-forming powder according to claim 1, wherein the retardant is selected from the group consisting of hydroxypropyl methyl cellulose acetate or succinate, hydroxypropyl methyl cellulose, hydroxypropyl cellulose, ethyl cellulose, hydroxyethyl cellulose, carboxymethyl cellulose sodium, polyvinyl alcohols, hydrocolloids such as: pectins, alginates, guar gum, xanthan gum, gum Arabic, agar, dextrin, carragheenan, polyethylene oxide, carbomers, polymers and copolymers of acrylic acid, of methyl methacrylate, of polyvinyl acetate, of carboxymethyl acetate and mixtures thereof.

11. Film-forming powder according to claim 1, wherein the bioerodible diluent is selected from the group consisting of calcium or sodium carbonate or bicarbonate, sucrose, mannitol, xylitol, sorbitol, lactose, cellulose or microcrystalline cellulose powder, starch and its derivatives, dibasic calcium phosphate, tribasic calcium phosphate, calcium sulphate, dextrates, dextrins, dextrose excipients, fructose, kaolin, lactitol and mixtures thereof.

12. Film-forming powder according to claim 1, wherein the penetration entrancer is selected from the group consisting of aliphatic fatty acid esters such as isopropyl myristate, fatty acids such as oleic acid; alcohols or polyols such as ethanol, propylene glycol and polyethylene glycol; components of essential oils and terpenic derivatives (such as eugenol, geraniol, nerol, eucalyptol, menthol); surfactants; moisturizers such as glycerin, urea; keratolytics such as alpha-hydroxy acids and mixtures thereof.

13. Film-forming powder according to claim 1, having a particle size of between 0.01 μm and 1000 μm, preferably between 0.1 μm and 100 μm and more preferably still between 1 μm and 50 μm.

14. Pharmaceutical, cosmetic or nutraceutical composition comprising a powder forming a film after application in situ according to any one of claim 1.

15. Composition according to claim 14, administered by the mucosal route.

16. Composition according to claim 15, administered through the buccal mucosa, the nasal mucosa or the vaginal mucosa.

17. Composition according to claim 14, administered by the transdermal route with local or systemic effect

18. Composition according to claim 14, being in pulverizable form.
